SEQUENCE LISTING (14 sequences)

SEQ ID NO: 1 - DNA, 100 bp, Homo sapiens
cgccttctgattgggacagccgtgggaaggacagttatgaaacgagtcagctggatgaccagagtgctgaaacccacagccacaagcagtccagattata

SEQ ID NO: 2 - DNA, 100 bp, Homo sapiens
cttcacagatatggtagccacggccaacaaccgtcagacctttgtcaactcggccatcaggtttctgcgcaaatacagctttgacggccttgaccttgac

SEQ ID NO: 3 - DNA, 100 bp, Homo sapiens
tggagacccaaatgtctcagaatgtatgtcccagaaacctgtggctgcttcaaccattgacagttttgctgctgctggcttctgcagacagtcaagctgc

SEQ ID NO: 4 - DNA, 100 bp, Homo sapiens
aaatcatgagggtgacgtagaattgagtcttccaggggactctatcagaactggaccatctccaagtatataacgatgagtcctcttaatgctaggagta

SEQ ID NO: 5 - protein, 106 aa, Homo sapiens
EELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGA

SEQ ID NO: 6 - protein, 118 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPS

SEQ ID NO: 7 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 345 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 345 aa, Homo sapiens; identical to SEQ ID NO: 10

SEQ ID NO: 12 - protein, 347 aa, Homo sapiens
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 5 aa, Homo sapiens
GGGGS

SEQ ID NO: 14 - protein, 11 aa, Homo sapiens
GTELSVRAKPS

(19) United States
(12) Patent Application Publication
(10) Pub. No.: US 2022/0289821 A1
(43) Pub. Date: Sep. 15, 2022

(54) BIOMARKERS FOR CD47 BLOCKADE THERAPY

(71) Applicant: TRILLIUM THERAPEUTICS INC, Mississauga (CA)

(72) Inventors: Lisa Danae Schultz Johnson, Etobicoke (CA); Mithunah Krishnamoorthy, Toronto (CA); Sandra Lauren Blitz, Kelowna (CA)

(21) Appl. No.: 17/297,353

(22) PCT Filed: Nov. 28, 2019
(86) PCT No.: PCT/CA2019/051699

§ 371 Date: May 26, 2021

Related U.S. Application Data
(60) Provisional application No. 62/772,876, filed on Nov. 29, 2018.

(51) Int. Cl. C07K 14/705; G01N 33/574; A61P 35/02

(52) U.S. Cl. CPC C07K 14/70596; G01N 33/57407; G01N 33/57488; A61P 35/02

(57) ABSTRACT

Subjects responsive to a CD47 blocking agent, such as SIRPαFc, exhibit an elevated level of expression of one or more markers. Accordingly, subjects with elevated levels of the markers are treated with CD47 blocking agents while subject that are not responsive and 5 do not have elevated levels of markers are not selected for treatment. The markers are selected from CHIT1, SPP1, FCγR3A and FCγR2A.

### FIELD

This invention relates to therapies that use inhibitors of the interaction between CD47 and SIRPα. More particularly, the invention relates to diagnostic and prognostic methods and means that are useful to identify subjects more likely to respond to a CD47 blocking agent.

### BACKGROUND

CD47 is an immune checkpoint that binds to signal regulatory protein alpha (SIRPα) and delivers a “do not eat” signal to suppress macrophage phagocytosis. Tumor cells frequently overexpress CD47 to evade macrophage-mediated destruction. Trillium's U.S. Pat. No. 9,969,789 describes a protein drug that inhibits the interaction between CD47 and SIRPα. This CD47 blocking agent is a form of human SIRPα that incorporates a particular region of its extracellular domain linked with a particularly useful form of an IgG1-based Fc region. A related form of SIRPα having an IgG4-based Fc region is also described. In these forms, SIRPαFc shows dramatic effects on the viability of cancer cells that present with a CD47+ phenotype. The effect is seen particularly in blood cancers as well as solid tumours. A soluble form of SIRP having significantly altered primary structure and enhanced CD47 binding affinity is described in WO2013/109752.

Other CD47 blocking agents have been described in the literature and these include various CD47 antibodies (see for instance Stanford's U.S. Pat. No. 8,562,997, and InhibRx′ WO2014/123580), each comprising different antigen binding sites but having, in common, the ability to compete with endogenous SIRPα for binding to CD47, thereby to allow interaction with macrophages and, ultimately, to increase the rate of CD47+ cancer cell depletion. These CD47 antibodies have activities in vivo that are quite different from those intrinsic to SIRPα-based drugs. The latter, for instance, display negligible binding to red blood cells whereas the opposite property in CD47 antibodies creates a need for strategies that accommodate the drug “sink” that follows administration.

Still other agents are proposed for use in blocking the CD47/SIRPα axis. These include CD47Fc fusion proteins (see Viral Logic's WO2010/083253), and SIRPα antibodies as described in UHN's WO2013/056352, Stanford's WO2016/022971, Eberhard's U.S. Pat. No. 6,913,894, and elsewhere.

The CD47 blockade approach in anti-cancer drug development shows great promise. It would be useful to provide methods and means for improving the effect of these drugs, and in particular for directing the use of the CD47 blocking agents, especially those that incorporate SIRPα.

To advance therapeutic applications of CD47 blocking agents, it would be useful to provide a basis for identifying and selecting subjects for treatment. More particularly, it would be helpful to provide a method whereby subjects most likely to respond favourably to treatment with a CD47 blocking agent could be identified, and then selected for subsequent or continued therapy.

### SUMMARY

It has now been determined that subjects responsive to a CD47 blocking agent will exhibit an elevated level of expression of one or more biomarkers or marker genes. The biomarker or marker gene is preferably selected from one that encodes osteopontin (SPP1), and/or encodes chitotriosidase (CHIT1) and/or encodes an Fc gamma receptor type that can be Type 3a (FcγR3a) and/or Type 2a (FcγR2a). This elevation in gene expression is seen in samples obtained from patients that have been dosed with a CD47 blocking agent. The present method accordingly permits the selection of subjects that are responsive to commencement or continuation therapy, particularly with a CD47 blocking agent that is a SIRPαFc fusion protein. Other subjects that are not responsive, as indicated by the absence of elevated gene expression, can be withdrawn from continued CD47 blockade therapy, and be prescribed a different course of therapy.

In one aspect, the biomarker is a marker gene, or is an expression product of that marker gene, such as an RNA transcript or protein that is derived from that marker gene.

In accordance with one aspect, there is provided a method of predicting responsiveness to therapy with a CD47 blocking agent, the method comprising determining, in a sample of CD47+ cancer obtained from a subject requiring treatment, the level of expression of one or more of the marker genes selected from SPP1, CHIT1, FCγR2A and FCγR3A following treatment with the agent, and comparing that expression level to a normal level thereof, whereby an increase in the level of a marker gene expression predicts that the cancer cell in the subject is responsive to therapy with a CD47 blocking agent.

In a related aspect, there is provided a method for identifying a subject responsive to treatment with a CD47 blocking agent (a responder), the method comprising determining the level of expression of one or more of the marker genes SPP1, CHIT1, FcγR2a and FcγR3a, whereby the subject is identified as a CD47 blocking agent responder when expression of at least one marker gene is elevated in response to treatment with said agent, relative to a normal level thereof.

In a further aspect, there is provided a treatment method comprising selecting for treatment a subject identified by the present method, and treating that subject with a CD47/SIRP blocking agent. In particular there is provided a method for treating a subject with a CD47 blocking agent, comprising testing a sample obtained from the subject to determine the expression level of one or more of the marker genes SPP1, CHIT1, FCγR2A and FCγR3A, and administering the CD47 blocking agent to the subject having an elevation in the expression level of at least one of these marker genes.

Also, there is provided the use of a CD47 blocking agent in a subject determined to have a cancer that responds to the agent with an elevated expression level of a marker gene selected from SPP1, CHIT1, FcγR2a and FcγR3a.

The detection methods used to quantify gene expression levels can be any of those in standard use for this purpose. The entity detected by these methods can be the messenger RNA (mRNA) translation of, or the protein expression products of, the noted marker genes, or any unique fragment thereof.

In another of its aspects, the present invention provides a kit useful in predicting patient response to therapy with a CD47 blocking agent, the kit comprising at least one reagent useful in determining the expression level of a marker gene, and instructions for the use thereof in the present methods. The reagents can include nucleic acid primers useful to amplify DNA or RNA obtained from a subject having or suspected of being at risk for cancer, e.g. from a tumour of that subject, wherein the primers have a nucleic acid sequence adapted to amplify a gene encoding CHIT1 and SPP1 and optionally FCγR2A and FCγR3A or an antibody to the marker gene-encoded protein, together with instructions for the use thereof in determining expression level of at least one of those genes.

In embodiments, the marker gene is a gene that encodes an Fcγ receptor, and is suitably either or both of FcγR2a and FcγR3a. In other embodiments, the marker gene is selected from FcγR2a, FcγR3a, CHIT1 and combinations thereof.

In embodiments, the CD47 blocking agent is a SIRP-based drug, such as SIRPαFc. In this drug, the Fc region desirably has effector function. The Fc region can preferably have an IgG1 isotype or an IgG4 isotype.

The present method is most usefully applied to identify those cancers, and subjects presenting with cancer, that will continue to respond to CD47 blocking agent therapy. The present method can equally reveal cancers that are predicted not to respond to such therapy, in that the target tissue does not reveal an elevated marker gene expression after administration of a CD47 blocking agent. This will indicate that a different therapeutic approach should be pursued.

These and other aspects of the present invention are now described in greater detail with reference to the accompanying drawings in which:

### BRIEF DESCRIPTION OF THE DRAWINGS

FIG. 1: shows volcano plots of gene expression data using Nanostring's PanCancer Immune Profiling panel comparing fold changes from baseline to maximum induration in CTCL patients with ≥50% decrease in CAILS (left) or <50% decrease in :CAILS (right). Fold change is indicated on the x-axis, p-value on the y-axis;

FIG. 2: A comparison of CHIT1, FCγR3A, and SPP1 fold-change from baseline to maximum induration for individual patients was plotted to determine the relationship between reduction in CAILS and gene upregulation. In all cases, decreased CAILS, indicating decreased tumor burden, correlates with increased expression. As shown, increased expression of CHIT1, FCγR3A, and SPP1 at maximum induration correlate with decreased CAILS, i.e., improved efficacy.

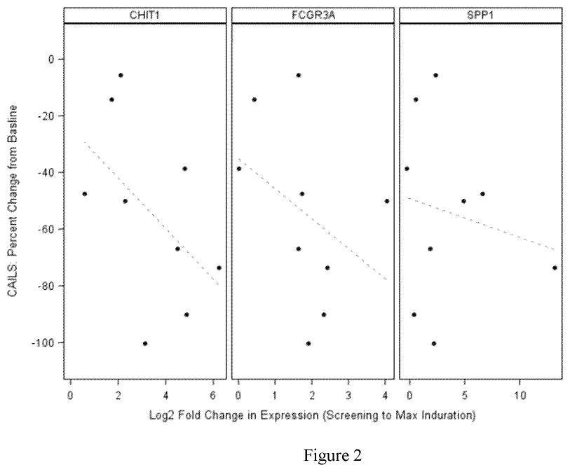

FIG. 3 reveals that osteopontin is produced in a SIRPαFc-dose dependent manner and correlates with the extent of phagocytosis. Phagocytosis is shown on the left y-axis with solid lines and filled in circles; osteopontin is shown on the right y-axis with dotted lines and unfilled circles.

FIG. 4 shows the FCγR2A fold-change from baseline to maximum induration for individual patients was plotted to determine the relationship between reduction in CAILS and gene upregulation. In all cases, decreased CAILS, indicating decreased tumor burden, correlates with increased expression of FCγR2A. As shown, increased expression at maximum induration correlates with decreased CAILS, i.e., improved efficacy.

### DETAILED DESCRIPTION

The present invention provides an improved method for treating a subject presenting with CD47+ disease cells such as cancer cells and tumours that have a CD47+ phenotype. In this method, subjects or biopsies therefrom receive a CD47 blocking agent such as SIRPαFc. Subjects are then selected for further treatment if testing reveals an increase in marker gene expression. Assessed for this purpose are the marker genes and/or the expression products of one or more marker genes selected from SPP1, CHIT1, FCγR2A, and FCγR3A.

As a result of testing to reveal the level of these marker genes or marker gene products, the present method enables drug therapy to be commenced or continued selectively in a particular group of subjects who would benefit most from such therapy.

The method can be applied either by testing a biopsy that has been treated ex vivo with a blocking agent, or by testing a biopsy obtained from a subject that has been dosed with a blocking agent. In either case, marker gene expression is tested after the sampled cancer is treated with the blocking agent, and is compared with a control such as an untreated sample counterpart, or a pre-treated sample counterpart.

In embodiments of this method, subjects receive a CD47 blocking agent such as SIRPαFc. Subjects are selected for treatment if their cancer tissue exhibits an increase in the presence or level of an expression product from one or more marker genes selected from SPP1, CHIT1, FCγR2A, and FCγR3A, Subjects being treated are selected for continuing therapy if marker gene elevation results from administration of a CD47 blocking agent.

In embodiments, the marker gene is selected from SPP1, CHIT1, FCγR2A and FCγR3A. These expression products of the marker genes influence macrophage recruitment to the cancer site, and thus are beneficial in the mechanism by which CD47 blockade controls CD47+ cancer. In one embodiment, the marker gene is SPP1. In another embodiment, the marker gene is CHIT1. In a further embodiment, the marker gene is FCγR2A. In another embodiment, the marker gene is FCγR3A. Combinations of two or more maker genes/gene products can be used in the determination.

The identifying reference numbers for the gene and the protein of each marker gene is set out below in Table 1, and full details of these sequences are incorporated herein by reference.

TABLE 1

| Gene Name | Protein Name | |
|---|---|---|
| Sequence | UniProtKB/SwissProt | Function |
| SPP1 | Osteopontin | Produced by Mphages and T cells; |
| Entrez 6696 | P10451 | matures DCs; upregulates IL-12; |
| | | inhibits IL-10 |
| CHIT1 | Chitotriosidase | Secreted by |
| Entrez 1118 | Q13231 | activated macrophages |
| FcγR2a | CD32a | Low affinity Fc binder. Elicits |
| NCBI gene | P12318 | phagocytosis and cytotoxicity by |
| 2212 | | macrophages |
| FcγR3a | CD16a | Elicits strong cytotoxicity and |
| Entrez 2214 | P08637 | cytokine production by NK cells |

In all embodiments, the feature of interest is an elevation in the level of an expression product from a marker gene, where the expression product is, for instance, protein or RNA produced via that gene, e.g., expressed from that marker gene. An “elevation” refers to an increase in the amount of the gene expression product in a tissue treated with a CD47 blocking agent relative to a healthy normal tissue counterpart (baseline) or to an untreated sample counterpart. The elevation of interest is also an increase in the amount of gene expression product in a subject before treatment with a CD47 blocking agent or during treatment with a CD47 blocking agent. That is, measuring gene expression levels before and during treatment is helpful in deciding whether the subject is a candidate for commencement or continuation of CD47 blockade therapy. Values for some marker genes are shown below:

TABLE 2

| | Log Fold | |
|---|---|---|
| Gene | Change Baseline to | |
| Name | Maximum Induration | p-value |
| CHIT1 | 4.195555341 | 0.003599995 |
| FCGR3A | 2.454144182 | 0.004307146 |
| FCGR2A | 1.69090015 | 0.009679458 |

For SPP1, the value at MI is 4.665 and p value is 0.1234.

As a result of screening to determine the level of marker gene expression, and administering the CD47 blocking agent to those subjects exhibiting gene expression levels higher than baseline, the present method enables drug therapy to be applied to, or continued selectively to, a particular group of subjects who would benefit most from such therapy.

The increase in marker gene expression is meaningful when the values for the subject after injection versus baseline have a difference that is statistically significant or relevant for the proposed mechanism of action.

The level of a marker gene can be determined using various biological samples, such as blood and other liquids of biological origin, solid tissue samples such as a biopsy specimens and tissue cultures or cells derived or isolated therefrom. The sample can have been manipulated, such as by treatment with reagents; washed; or enriched for certain cell populations, such as cancer cells. The sample can be one that is enriched for particular types of molecules, e.g., nucleic acids such as RNA, polypeptides, etc. Samples include clinical samples. These sample types include tissue obtained by surgical resection or by biopsy, cells in culture, cell supernatants, cell lysates, organs, bone marrow, blood, plasma, serum, an aspirate, and the like. A sample can include biological fluids derived from cells (e.g., a cancerous cell, an infected cell, etc.), a sample comprising polynucleotides and/or polypeptides that is obtained from such cells (e.g., a cell lysate or other cell extract comprising polynucleotides and/or polypeptides).

A sample is obtained by physical extraction or isolation of a sample from a subject. Methods for isolating samples, e.g., blood, serum, plasma, biopsy, aspirate, etc., are well known.

The “level”, or expression level of a marker gene product, which may be an RNA, a protein, etc., in a sample is measured (i.e., “determined”). By “expression level” (or “level”) it is meant the level of gene product (e.g. the absolute and/or normalized value determined for the RNA expression level of a marker gene or for the expression level of the encoded polypeptide, or the concentration of the protein in a biological sample). The term “gene product” or “expression product” are used herein to refer to the RNA transcription products (RNA transcripts, e.g. mRNA, an unspliced RNA, a splice variant mRNA, and/or a fragmented RNA) of a marker gene, including mRNA, and the polypeptide translation products of such RNA transcripts. A gene product can be, for example, an unspliced RNA, an mRNA, a splice variant mRNA, a microRNA, a fragmented RNA, a polypeptide, a pre-polypeptide, a propolypeptide, a prepropolypeptide, a post-translationally modified polypeptide, a splice variant polypeptide, etc.

The terms “determining” and “testing” are used interchangeably herein to refer to any form of measurement, and include determining if an element is present or not. These terms include both quantitative, semi-quantitative and/or qualitative determinations. For example, “testing” can be determining whether the expression level is less than or “greater than or equal to” a particular threshold, (the threshold can be pre-determined or can be determined by assaying a control sample). On the other hand, assaying to determine the expression level can involve determining a quantitative value (using any convenient metric) that represents the level of expression (i.e., expression level, e.g., the amount of protein and/or RNA, e.g., mRNA) of a particular marker gene. The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of a marker gene can be compared to the expression level of one or more additional genes (e.g., nucleic acids and/or their encoded proteins) to derive a normalized value that represents a normalized expression level.

For measuring RNA levels, the amount or level of an RNA, such as an RNA transcript, in the sample is determined. In some instances, the expression level of one or more additional RNAs may also be measured, and the level of marker gene expression compared to the level of the one or more additional RNAs to provide a normalized value for marker gene expression level. Any convenient protocol for evaluating RNA levels may be employed wherein the level of one or more RNAs in the assayed sample is determined. Distinctive marker gene fragments can also be a detection target. These are regions of the marker gene that are unique to that gene, so that amplification, and the conditions selected for amplification, yields an amplicon that is representative of that marker gene only. The distinctive fragment can be a unique portion of the protein-encoding region of the marker gene, such as an extracellular region, or a portion residing in the upstream elements that regulate expression of that gene, or a portion residing in the downstream region that regulates termination of transcription, among other regions.

Many useful approaches are known for measuring RNA e.g., mRNA, expression levels in a sample and any of these methods can be used. These methods include: hybridization-based methods such as Northern blotting, array hybridization (e.g., microarray); in situ hybridization; in situ hybridization followed by FACS; and the like; RNAse protection assays; PCR-based methods including reverse transcription PCR (RT-PCR), quantitative RT-PCR (qRT-PCR), real-time RT-PCR; nucleic acid sequencing methods, e.g., massive parallel high throughput sequencing, such as Illumina's reversible terminator method, Roche's pyrosequencing method, Life Technologies' sequencing by ligation (the SOLID platform), Life Technologies' Ion Torrent platform; and the like.

The raw sample can be tested. In the alternative, nucleic acid of the biological sample is amplified (e.g., by PCR) prior to testing. As such, techniques such as PCR (Polymerase Chain Reaction), RT-PCR (reverse transcriptase PCR), qRT-PCR (quantitative RT-PCR, real time RT-PCR), and the like can be used before hybridization methods and/or the sequencing methods.

For measuring mRNA levels, the starting material is typically total RNA or poly A+RNA isolated from a sample, e.g., a suspension of cells from a peripheral blood sample, a bone marrow sample, etc., or from a homogenized tissue, e.g. a homogenized biopsy sample, an aspirate, a homogenized paraffin- or OCT-embedded sample, etc.). RNA isolation can be performed using a purification kit, buffer set and protease from commercial manufacturers, according to the manufacturer's instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

Various ways of determining/measuring mRNA levels are known in the art, e.g. as employed in the field of differential gene expression analysis. One protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, and complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference. In these methods, an array of “probe” nucleic acids that includes a probe for each of the marker gene is contacted with target nucleic acids as described above. Contact is carried out under hybridization conditions, e.g., stringent hybridization conditions, and unbound nucleic acid is then removed. Stringent assay conditions use binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity.

The resultant pattern of hybridized nucleic acid provides information regarding expression for each of the marker genes that have been probed, e.g., at least one or more of SPP1, CHIT1 and FCγR3A and FCγR2A, where the expression information is in terms of whether or not the gene is expressed and, typically, at what level, where the expression data.

Non-array-based methods for quantitating the level of one or marker gene products in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan® RT-PCR, MassARRAY® System, BeadArray® technology, and Luminex technology; and those that rely upon hybridization of probes to filters, e.g. Northern blotting and in situ hybridization.

For measuring protein levels, as expression products of the marker genes, the amount or level of a polypeptide in the biological sample is determined. In some embodiments, concentration is a relative value measured by comparing the level of one protein relative to another protein, or the level of the protein in one sample versus the level of the same protein in a different sample. An enhanced or elevated level of gene expression is relevant when it has statistical significance, and especially when its increase over base line has a p value greater than 0.5 such as greater than 0.01 or better. Elevation is evident when the marker gene expression level is about 50% greater, e.g. at least one-fold, two-fold, three-fold greater than marker gene expression baseline.

In some cases, the cells are removed from the biological sample, e.g., via centrifugation, via adhering cells to a dish or to plastic, etc., before testing. In some cases, the intracellular protein level is measured by lysing the removed cells of the biological sample to measure the level of protein in the cellular contents. In some cases, both the extracellular and intracellular levels of protein are measured by separating the cellular and fluid portions of the biological sample (e.g., via centrifugation), measuring the extracellular level of the protein by measuring the level of protein in the fluid portion of the biological sample, and measuring the intracellular level of protein by measuring the level of protein in the cellular portion of the biological sample (e.g., after lysing the cells). In some cases, the total level of protein (i.e., combined extracellular and intracellular protein) is measured by lysing the cells of the biological sample to include the intracellular contents as part of the sample.

In some embodiments, the presence, concentration or level of one or more additional proteins may also be measured, and marker gene-expressed protein levels are compared to the level of the one or more additional proteins to provide a normalized value for the maker gene product/protein concentration. Any convenient protocol for evaluating protein levels may be employed wherein the level of one or more proteins in the assayed sample is determined.

One representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzothiazoline)-6-sulfonic acid (ABTS) and H2O2, in the case of a peroxidase label. Quantitation is then achieved by measuring the degree of color generation, e.g., using a visible spectrum spectrophotometer.

The ELISA or EIA format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected by removing a bead, emptying or diluting a reservoir such as a microtiter plate well, or rinsing a bead, particle, chromatographic column or filter with a wash solution or solvent.

Non-ELISA based-methods for measuring the levels of one or more proteins in a sample may be employed. Representative exemplary methods include Western blotting, proteomic arrays, xMAP™ microsphere technology (e.g., Luminex technology), immunohistochemistry, flow cytometry, and the like as well as non-antibody-based methods (e.g., mass spectrometry).

For comparison, the level of the same marker in a different sample can also be determined. The different sample can be taken from a subject that is healthy and tumour-free, or from a subject that is selected to undergo CD47 blockade therapy but has yet to be so treated. Accordingly, the marker gene product level can be determined at intervals including pre-treatment, commencement of treatment, e.g., after first dose, and during treatment and post treatment.

In one specific embodiment, the level of a marker gene expression product is determined using the NanoString® approach described in the examples herein. In this approach, RNA from a sample taken from a subject is assayed using multiplex gene expression analysis with 770 genes from 24 different immune cell types including tumour cell infiltrating lymphocytes, common checkpoint inhibitors, CT antigens, and genes covering both the adaptive and innate immune response. To detect and quantify the level of marker gene expression, this approach identifies sample-borne RNA using hybridizing probes having the sequences noted below:

For SPP1 NM_000582.2
[SEQ ID No. 1]
CGCCTTCTGATTGGGACAGCCGTGGGAAGGACAGTTATGAAACGAGTCAG
CTGGATGACCAGAGTGCTGAAACCCACAGCCACAAGCAGTCCAGATTATA;
For CHIT1 NM_003465.2
[SEQ ID No. 2]
CTTCACAGATATGGTAGCCACGGCCAACAACCGTCAGACCTTTGTCAACT
CGGCCATCAGGTTTCTGCGCAAATACAGCTTTGACGGCCTTGACCTTGAC;
For FcγR2a NM_021642.3
[SEQ ID No. 3]
TGGAGACCCAAATGTCTCAGAATGTATGTCCCAGAAACCTGTGGCTGCTT
CAACCATTGACAGTTTTGCTGCTGCTGGCTTCTGCAGACAGTCAAGCTGC;
and
For FcγR3a NM_000569.6
[SEQ ID No. 4]
AAATCATGAGGGTGACGTAGAATTGAGTCTTCCAGGGGACTCTATCAGAA
CTGGACCATCTCCAAGTATATAACGATGAGTCCTCTTAATGCTAGGAGTA

Thus, in one aspect, there is provided a method useful to identify a cancer subject that will respond to treatment with a CD47 blocking agent, the method comprising identifying for treatment with the CD47 blocking agent the cancer subject that responds to treatment with the agent with an elevated marker gene expression level, wherein the marker gene is SPP1, CHIT1, FcγR2a or FcγR3a.

In another aspect there is provided a method of predicting responsiveness to treatment of a cancer with a CD47 blocking agent, treating a subject with the blocking agent, and then determining the level of expression of one, two or all of the marker genes CHIT1, FCγR2A, FCγR3A and SPP1 in a sample of that cancer obtained from that subject wherein elevated CHIT1 or FCγR2A or FCγR3A or SPP1 expression predicts the cancer will respond to or will continue to respond to therapy with a CD47 blocking agent. In embodiments, the method is applied to a subject that has already been dosed at least once with CD47 blocking agent, and marker gene response to that dose is determined. Subject that exhibit an increase in marker gene expression are identified for continuing treatment. Subjects that fail to exhibit an increase in marker gene expression are withdrawn from such therapy and can prescribed a different therapy.

There is also provided a method for treating a subject with a CD47 blocking agent, comprising determining in a sample obtained from the subject the expression level of one or more of the marker genes CHIT1, FCγR2A, FCγR3A and SPP1, and administering the CD47 blocking agent to the subject in which the level of expression of a marker gene is elevated by administration of the CD47 blocking agent.

A wide variety of CD47 blocking agents are useful in the present method. As used herein, the term “anti-CD47 agent” or “CD47-blocking agent” or “CD47 blockade drug” refers to any agent that reduces the binding of CD47 (e.g., on a target cell) to SIRPα (e.g., on a phagocytic cell). Non-limiting examples of suitable anti-CD47 reagents include SIRPα reagents, including without limitation high affinity SIRPα polypeptides, anti-SIRPα, antibodies, soluble CD47 polypeptides, and anti-CD47 antibodies or antibody fragments. In some embodiments, a suitable anti-CD47 agent (e.g. an anti-CD47 antibody, a SIRPα reagent, etc.) specifically binds CD47 to reduce the binding of CD47 to SIRPα.

In some embodiments, a suitable anti-CD47 agent (e.g., an anti-SIRPα, antibody, a soluble CD47 polypeptide, etc.) specifically binds SIRPα to reduce the binding of CD47 to SIRPα. A suitable anti-CD47 agent that binds SIRPα does not activate SIRPα.

The term “CD47+” is used herein with reference to the phenotype of cells targeted for treatment with a CD47 blocking agent. Cells that are CD47+ can be identified by flow cytometry using CD47 antibody as the affinity ligand. Labeled CD47 antibodies are available commercially for this use (for example, clone B6H12 is available from Santa Cruz Biotechnology). The cells examined for CD47 phenotype can be standard tumour biopsy samples including particularly liquid and tissue samples taken from the subject suspected of harbouring CD47+ cancer cells. CD47 disease cells of particular interest as targets for therapy with the present combination are those that “over-express” CD47. These CD47+ cells typically are disease cells, and present CD47 at a density on their surface that exceeds the normal CD47 density for a cell of a given type. CD47 overexpression will vary across different cell types, but is meant herein to refer to any CD47 level that is determined, for instance by flow cytometry or by immunostaining or by gene expression analysis or the like, to be greater than the level measurable on a counterpart cell having a CD47 phenotype that is normal for that cell type.

As used herein, a “CD47 blocking agent” can be any drug or agent that interferes with and dampens or blocks signal transmission that results when CD47 interacts with macrophage-presented SIRPα. The CD47 blocking agent is an agent that inhibits CD47 interaction with SIRPα. The CD47 blocking agent is preferably an agent that binds CD47 and blocks its interaction with SIRPα. The CD47 blocking agent can be an antibody or antibody-based antagonist of the CD47/SIRPα signaling axis, such as an antibody that binds CD47 and blocks interaction of CD47 with SIRPα.

Desirably, but not essentially, the CD47 blocking agent comprises a constant region, i.e., an Fc region, that can be bound by macrophages that are activated to destroy cells to which the CD47 blocking agent is bound, such as cancer cells. The CD47 blocking agent Fc region preferably has effector function, and is derived preferably from either IgG1 or IgG4 including IgG4(S228P). In the alternative, the Fc region can be one that is altered by amino acid substitution to change effector function, e.g., to an inactive state.

CD47-binding forms of human SIRPα are the preferred CD47 blocking agents for use in the combination herein disclosed. These drugs are based on the extracellular region of human SIRPα. They comprise at least a part of the extracellular region sufficient to confer effective CD47 binding affinity and specificity. So-called “soluble” forms of SIRPα, lacking the membrane anchoring property in SIRPα, are useful and include those referenced in Novartis' WO 2010/070047, Stanford's WO2013/109752, Merck's WO2016/024021 and Trillium's WO2014/094122 and Merck's WO2016/024021.

The SIRPαFc drug useful in the present method can be a monomeric, homodimeric or heterodimeric form of a single chain polypeptide comprising an Fc region of an antibody and a CD47-binding region of human SIRPα.

In preferred embodiments, the SIRPαFc polypeptide has the properties discussed below. More particularly, the polypeptide suitably comprises a CD47-binding part of human SIRPα protein in a form fused directly, or indirectly, with an antibody constant region, or Fc (fragment crystallisable). Unless otherwise stated, the term “human SIRPα” as used herein refers to a wild type, endogenous, mature form of human SIRPα. In humans, the SIRPα protein is found in two major forms. One form, the variant 1 or V1 form, has the amino acid sequence set out as NCBI RefSeq NP 542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of binding with, and triggering signal transduction through CD47. The present invention is directed most particularly to the drug combinations that include the V2 form of SIRPα.

In the present drug combination, useful CD47 blocking agents are SIRPαFc fusion polypeptides that comprise at least one of the three so-called immunoglobulin (Ig) domains within the extracellular region of human SIRPα. More particularly, the present SIRPαFc polypeptides preferably incorporate residues 32-137 of human SIRPα (a 106-mer), which constitute and define the IgV domain of the V2 form according to current nomenclature. This SIRPα sequence, shown below, is referenced herein as SEQ ID No. 5.

[SEQ ID No. 5]
EELQVIQPDKSVSVAAGESAILHCTVTSLIPVGPIQWFRGAGPARELIYNQ
KEGHFPRVTTVSESTKRENIVIDFSISISNITPADAGTYYCVKFRKGSPDT
EFKSGA

In a preferred embodiment, the SIRPαFc fusion protein incorporates the IgV domain as defined by SEQ ID No. 5, and additional, flanking residues contiguous within the wild type human SIRPα sequence. This preferred form of the IgV domain, represented by residues 31-148 of the V2 form of human SIRPα, is a 118-mer having SEQ ID No. 6 shown below:

[SEQ ID No. 6]
EEELQVIQPDKSVSVAAGESAMECTVTSLIPVGPIQWFRGAGPARELIYNQ
KEGHFPRVTTVSESTKRENMDFSISISNITPADAGTYYCVKFRKGSPDTEF
KSGAGTELSVRAKPS

The Fc region of the SIRPαFc fusion polypeptide preferably does have effector function. Fc refers to “fragment crystallisable” and represents the constant region of an IgG antibody comprised principally of the heavy chain constant region and components within the hinge region. Suitable Fc components thus are those having effector function. An Fc component “having effector function” is an Fc component having at least some effector function, such as at least some contribution to antibody-dependent cellular cytotoxicity or some ability to fix complement. Also, the Fc will at least bind to one or more types of Fc receptor. These properties can be revealed using assays established for this purpose. Functional assays include the standard chromium release assay that detects target cell lysis. By this definition, an Fc region that is wild type IgG1 or IgG4 has effector function, whereas the Fc region of a human IgG4 mutated to eliminate effector function, such as by incorporation of an alteration series that includes Pro233, Val234, Ala235 and deletion of Gly236 (EU), is considered not to have effector function. In a preferred embodiment, the Fc is based on human antibodies of the IgG1 isotype. In an alternative embodiment, the Fc is based on the IgG4 isotype, and includes the Pro228Ser variation. The Fc region of these antibodies will be readily identifiable to those skilled in the art. In embodiments, the Fc region includes the lower hinge-CH2-CH3 domains.

In a specific embodiment, the Fc region is based on the amino acid sequence of a human IgG1 set out as P01857 in UniProtKB/Swiss-Prot, residues 104-330, and has the amino acid sequence shown below and referenced herein as SEQ ID No. 7:

[SEQ ID No. 7]
DKTHTCPPCPAPELLGGPSVFLEPPKPKDTLMISRTPEVTCVVVDVSHEDP
EVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVFS
CSVMHEALHNHYTQKSLSLSPGK*

Thus, in embodiments, the Fc region has either a wild type or consensus sequence of an IgG1 constant region. In alternative embodiments, the Fc region incorporated in the fusion protein is derived from any IgG1 antibody having a typical effector-active constant region. The sequences of such Fc regions can correspond, for example, with the Fc regions of any of the following IgG1 sequences (all referenced from GenBank), for example: BAG65283 (residues 242-473), BAC04226.1 (residues 247-478), BAC05014.1 (residues 240-471), CAC20454.1 (residues 99-320), BAC05016.1 (residues 238-469), BAC85350.1 (residues 243-474), BAC85529.1 (residues 244-475), and BAC85429.1 (residues (238-469).

In other embodiments, the Fc region has a sequence of a wild type human IgG4 constant region. In alternative embodiments, the Fc region incorporated in the fusion protein is derived from any IgG4 antibody having a constant region with effector activity that is present but, naturally, is less potent than the IgG1 Fc region. The sequences of such Fc regions can correspond, for example, with the Fc regions of any of the following IgG4 sequences: P01861 (residues 99-327) from UniProtKB/Swiss-Prot and CAC20457.1 (residues 99-327) from GenBank.

In a specific embodiment, the Fc region is based on the amino acid sequence of a human IgG4 set out as P01861 in UniProtKB/Swiss-Prot, residues 99-327, and has the amino acid sequence shown below and referenced herein as SEQ ID No. 8:

[SEQ ID No. 8]
ESKYGPPCPSCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK

In embodiments, the Fc region incorporates one or more alterations, usually not more than about 5 such alterations, including amino acid substitutions that affect certain Fc properties. In one specific and preferred embodiment, the Fc region incorporates an alteration at position 228 (EU numbering), in which the serine at this position is substituted by a proline (S228P), thereby to stabilize the disulfide linkage within the Fc dimer. Other alterations within the Fc region can include substitutions that alter glycosylation, such as substitution of Asn297 by glycine or alanine; half-life enhancing alterations such as T252L, T253 S, and T256F as taught in U.S. 62/777,375, and many others including the 409 position. Particularly useful are those alterations that enhance Fc properties while remaining silent with respect to conformation, e.g., retaining Fc receptor binding.

In a specific embodiment, and in the case where the Fc component is an IgG4 Fc, the Fc incorporates at least the S228P mutation, and has the amino acid sequence set out below and referenced herein as SEQ ID No. 9:

[SEQ ID No. 9]
ESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGK

The CD47 blocking agent used in the combination is thus a SIRPαFc fusion protein useful to inhibit binding between human SIRPα and human CD47, thereby to inhibit or reduce transmission of the signal mediated via SIRPα-bound CD47, the fusion protein comprising a human SIRPα component and, fused therewith, an Fc component, wherein the SIRPα component comprises or consists of a single IgV domain of human SIRPα V2 and the Fc component is the constant region of a human IgG, wherein the constant region preferably has effector function.

In one embodiment, the fusion protein comprises a SIRPα component consisting at least of residues 32-137 of the V2 form of wild type human SIRPα, i.e., SEQ ID No. 5. In a preferred embodiment, the SIRPα component consists of residues 31-148 of the V2 form of human SIRPα, i.e., SEQ ID No. 6. In another embodiment, the Fc component is the Fc component of the human IgG1 designated P01857, and in a specific embodiment has the amino acid sequence that incorporates the lower hinge-CH2-CH3 region thereof i.e., SEQ ID No. 7.

In a preferred embodiment, therefore, the present method utilizes a CD47 blocking agent that is a SIRPαFc fusion polypeptide, as both an expressed single chain polypeptide and as a secreted dimeric fusion thereof (homodimer), wherein the fusion protein incorporates a SIRPα component having SEQ ID No. 5 and preferably SEQ ID No. 6 and, fused therewith, an Fc region having effector function and having SEQ ID No. 7. When the SIRPα component is SEQ ID No. 5, this fusion protein comprises SEQ ID No. 10, shown below:

[SEQ ID No. 10]
EEELQVIQPDKSVSVAAGESAILHCTVTSLIPVGPIQWFRGAGPARELIYN
QKEGHFPRVTTVSESTKRENMDFSISISNITPADAGTYYCVKFRKGSPDTE
FKSGAGTELSVRAKPSDKTHTCPPCPAPELLGGPSVFLFPPKPKDTLMISR
TPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYS
KLTVDKSRWQQGNVFSCSVMHEALHNHYTQKSLSLSPGK*

When the SIRPα component is SEQ ID No. 6, this fusion protein comprises SEQ ID No. 11, shown below:

[SEQ ID No. 11]
EEELQVIQPDKSVSVAAGESAILHCTVTSLIPVGPIQWFRGAGPARELIYN
QKEGHFPRVTTVSESTKRENMDFSISISNITPADAGTYYCVKFRKGSPDTE
FKSGAGTELSVRAKPSDKTHTCPPCPAPELLGGPSVFLFPPKPKDTLMISR
TPEVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYS
KLTVDKSRWQQGNVFSCSVMHEALHNHYTQKSLSLSPGK

In alternative embodiments, the Fc component of the fusion protein is based on an IgG4, and preferably an IgG4 that incorporates the S228P mutation. In the case where the fusion protein incorporates the preferred SIRPα IgV domain of SEQ ID No. 6, the resulting IgG4-based SIRPα-Fc protein has SEQ ID No. 12, shown below:

[SEQ ID No. 12]
EEELQVIQPDKSVSVAAGESAILHCTVTSLIPVGPIQWFRGAGPARELIYN
QKEGHFPRVTTVSESTKRENMDFSISISNITPADAGTYYCVKFRKGSPDTE
FKSGAGTELSVRAKPSESKYGPPCPPCPAPEFLGGPSVFLFPPKPKDTLMI
SRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFFL
YSRLTVDKSRWQEGNVFSCSVMHEALHNHYTQKSLSLSLGK

In preferred embodiment, the fusion protein comprises, as the SIRPα IgV domain of the fusion protein, a sequence that is SEQ ID No. 6. The preferred SIRPαFc is SEQ ID No. 11.

The SIRPα sequence incorporated within the CD47 blocking agent can be varied, as described in the literature. That is, useful substitutions within SIRPα will typically enhance binding affinity for CD47, and can include one or more of the following: L4V/I, V6I/L, A21V, V27I/L, 131T/S/F, E47V/L, K53R, E54Q, H56P/R, S66T/G, K68R, V92I, F94V/L, V63I, and/or F103V. Still other substitutions include conservative amino acid substitutions in which an amino acid is replaced by an amino acid from the same group. Also as noted, the SIRPα sequence can also be truncated or extended, so long as CD47 binding affinity is retained.

In the SIRPαFc fusion polypeptide, the SIRPα component and the Fc component are fused, either directly or indirectly, to provide a single chain polypeptide that is ultimately produced as a homodimer in which the single chain polypeptides are coupled through intrachain disulfide bonds formed between the Fc regions of individual single chain SIRPαFc polypeptides. The nature of the fusing region that joins the SIRPα region and the Fc is not critical. The fusion may be direct between the two components, with the SIRP component constituting the N-terminal end of the fusion and the Fc component constituting the C-terminal end. Alternatively, the fusion may be indirect, through a linker comprised of one or more amino acids, desirably genetically encoded amino acids, such as two, three, four, five, six, seven, eight, nine or ten amino acids, or any number of amino acids between 5 and 100 amino acids, such as between 5 and 50, 5 and 30 or 5 and 20 amino acids. A linker may comprise a peptide that is encoded by DNA constituting a restriction site.

The linker amino acids typically and desirably will provide some flexibility to allow the Fc and the SIRPα components to adopt their active conformations. Residues that allow for such flexibility typically are Gly, Asn and Ser, so that virtually any combination of these residues (and particularly Gly and Ser) within a linker is likely to provide the desired linking effect. In one example, such a linker is based on the so-called G45 sequence (Gly-Gly-Gly-Gly-Ser) (SEQ ID No. 13) which may repeat as (G4S)n where n is 1, 2, 3 or more, or is based on (Gly)n, (Ser)n, (Ser-Gly)n or (Gly-Ser)n and the like. In another embodiment, the linker is GTELSVRAKPS (SEQ ID No. 14). This sequence constitutes a SIRPα sequence that C-terminally flanks the IgV domain (it being understood that this flanking sequence could be considered either a linker or a different form of the IgV domain when coupled with the IgV minimal sequence described above). It is necessary only that the fusing region or linker permits the components to adopt their active conformations, and this can be achieved by any form of linker useful in the art.

The CD47 blocking agent can also be an antibody that specifically binds CD47, a suitable anti-CD47 antibody does not activate CD47 upon binding. Non-limiting examples of suitable antibodies include clones B6H12, 5F9, 8B6, and C3 (for example as described in WO 2011/143624, herein specifically incorporated by reference. Suitable anti-CD47 antibodies include fully human, humanized or chimeric versions of such antibodies. Humanized antibodies (e.g., hu5F9-G4) are especially useful for in vivo applications in humans due to their low antigenicity.

These gene markers are useful to identify cancers that will respond favourably to therapy with a CD47 blocking agent. By analogy, the gene markers are also useful to identify subjects who will respond to such therapy, i.e. subjects having a cancer that will respond favourably. Those subjects or cancers that “respond favourably” are those cancers or subjects that respond to administration of the inhibitor with improvements in the symptoms of the disease being treated. For instance, the response could manifest as an improvement in cancer cell or tumour properties or dynamics, such as a reduction in tumour growth rate, in cancer cell or tumour size or number, in cancer cell or tumour distribution and/or in overall cancer cell or tumour burden, for example, and/or as an extension of survival or an improvement in quality of life of the subject presenting with cancer.

In the present method, the subjects to whom the method is most appropriately applied are subjects, such as mammals including pets, horses, livestock, primates and particularly humans, presenting with cancer and particularly a CD47+ cancer including a CD47+ hematological cancer or a CD47+ solid cancer such as a malignant tumour. In the alternative, the subject can be one that presents with any disease that can be treated with a CD47 blocking agent. In embodiments, the disease is a blood cancer selected from a lymphoma, a leukemia or a myeloma, and can be further selected from Hodgkin's lymphoma, both indolent and aggressive non-Hodgkin's lymphoma, Burkitt's lymphoma, follicular lymphoma (small cell and large cell), promyelocytic leukemia, chronic and acute myeloid leukemia (AML), acute and chronic lymphoid leukemia, multiple myeloma (MM), giant cell myeloma, heavy-chain myeloma, and light chain or Bence-Jones myeloma as well as diffuse large B-cell lymphoma (DLCBL), T-cell acute lymphoblastic leukemia (T-ALL), and T-cell lymphoma including cutaneous T cell lymphoma (CTCL), mycosis fungoides and Sezary syndrome. The cancer is also referenced herein as a tumour or as a cancer cell. Other diseases include infection such as viral infection, as well as other diseases involving aberrant CD47 protein, which can include those mediated by altered CD47 protein gene expression or CD47 protein mutation or the like.

The prediction based on expression of the present maker genes that a given tumour will respond to CD47 blockade is particularly accurate when the cancer is a solid or at least palpable tumour or blood cancer, and one of those cancers identified herein, e.g., above. Solid cancers including lung, prostate, breast, bladder, colon, ovarian, glioblastoma, medulloblastoma, leiomyosarcoma, and head & neck squamous cell carcinomas, melanomas; etc.

In use, these CD47 blocking agents are formulated with a pharmaceutically acceptable carrier using standard practices and ingredients. The formulated drug will be administered parenterally such as by injection or infusion, or orally in the form of tablets, capsules liquids and the like. Dosing and dosing regimens will be standard for drugs in this same category.

In other specific embodiments, the cancer for which prediction of response is determined is one that is a hematological cancer and particularly one that is selected from the group consisting of Hodgkin's lymphoma, indolent and aggressive non-Hodgkin's lymphoma, Burkitt's lymphoma, follicular lymphoma, promyelocytic leukemia, chronic and acute myeloid leukemia, acute and chronic lymphoid leukemia, multiple myeloma (MM), giant cell myeloma, heavy-chain myeloma, and light chain or Bence-Jones myeloma, diffuse large B-cell lymphoma (DLCBL), cutaneous anaplastic large cell lymphoma (pcALCL), Sezary Syndrome, T-cell acute lymphoblastic leukemia (T-ALL), and T-cell lymphoma including particularly cutaneous T cell lymphoma (CTCL).

In a typical application of the present method, a subject presenting with a cancer having a CD47+ phenotype is recruited for treatment with, for instance, a SIRPαFc (G1) agent, and the pharmacokinetics, pharmacodynamics and antitumor activity of intralesional injections of SIRPαFc are studied in adult patients with, for instance, CTCL or relapsed/refractory (R/R) percutaneously accessible solid tumors, or mycosis fungoides (MF).

Following administration of a first dose of SIRPαFc or additional doses if desired, biopsied tissue is tested to determine whether expression of any one or more of the marker genes is at a level that is elevated relative to a pre-treatment level. If there is elevation in at least any one marker gene expression, then SIRPαFc therapy can continue since the subject is deemed a responder to CD47 blockade therapy.

A “dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of an anti-CD47 agent is an amount that is sufficient to palliate, ameliorate, stabilize, reverse, prevent, slow or delay the progression of the disease state (e.g., cancer or chronic infection) by increasing phagocytosis of a target cell (e.g., a target cell). Thus, a therapeutically effective dose of an anti-CD47 agent reduces the binding of CD47 on a target cell, to SIRPα on a phagocytic cell, at an effective dose for increasing the phagocytosis of the target cell.

An effective dose or a series of therapeutically effective doses would be able to achieve and maintain a serum level of anti-CD47 agent. A therapeutically effective dose of SIRPαFc agent can depend on the specific agent used, but is usually about 2 mg/kg body weight or more (e.g., about 2 mg/kg or more, about 4 mg/kg or more, about 8 mg/kg or more, about 10 mg/kg or more, about 15 mg/kg or more, about 20 mg/kg or more, about 25 mg/kg or more, about 30 mg/kg or more, about 35 mg/kg or more, or about 40 mg/kg or more), or from about 10 mg/kg to about 40 mg/kg (e.g., from about 10 mg/kg to about 35 mg/kg, or from about 10 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

A sub-therapeutic dose is a dose (i.e., an amount) that is not sufficient to effect the desired clinical results. For example, a sub-therapeutic dose of an anti-CD47 agent is an amount that is not sufficient to palliate, ameliorate, stabilize, reverse, prevent, slow or delay the progression of the disease state. In some cases, it is desirable to use a sub-therapeutic dose of an anti-CD47 agent as a primer agent, such as an agent intended to test the effect of the drug on marker gene expression levels. A sub-therapeutic dose of an anti-CD47 agent can depend on the specific agent used, but is generally less than about 10 mg/kg.

The term “continue treatment” (i.e., continue therapy) is used herein to mean that the planned or current course of treatment (e.g., continued administration of an anti-CD47 agent) is to continue, because the marker gene expression results show an elevation. “Altering therapy” means replacing current therapy with either no therapy or a different CD47 therapeutic or a different drug altogether.

The anti-CD47 agent can be administered to an individual any time after a pre-treatment biological sample is isolated from the individual. The anti-CD47 agent may be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment biological sample is isolated (or, when multiple pre-treatment biological samples are isolated, after the final pre-treatment biological sample is isolated).

Suitable anti-CD47 agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an anti-CD47 agent includes use in combination with another therapeutic agent (e.g., another anti-infection agent or another anti-cancer agent). Therapeutic formulations comprising one or more anti-CD47 agents of the invention are prepared for storage by mixing the anti-CD47 agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The anti-CD47 agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The anti-CD47 agent can be “administered” by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration.

An anti-CD47 agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

Compositions can be prepared as injectables, either as liquid solutions or suspensions; solid forms suitable for solution in, or suspension in, liquid vehicles can also be prepared. The preparation also can be encapsulated in liposomes or micro particles such as polylactide, polyglycolide, or copolymer for enhanced adjuvant effect, as discussed above. The agents can be administered as a depot injection or implant preparation which can be formulated for sustained or pulsatile release of the active ingredient. The pharmaceutical compositions are generally formulated as sterile, substantially isotonic and in full compliance with regulatory agencies.

Also provided are kits for use in the present methods. The subject kits include a tool e.g., a marker gene-hybridizing and optionally labeled oligonucleotide, or a PCR primer pair specific for a marker gene expression product such as RNA, or an antibody that specifically binds to a marker gene expressed protein, and the like) for determining the expression level of at least one marker gene. A kit can also include an anti-CD47 agent, such as SIRPαFc. An anti-CD47 agent can be provided in a dosage form (e.g., a therapeutically effective dosage form, e.g., stick pack, dose pack, etc.).

The kits may further include instructions for practicing the present methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium on which the information has been recorded. Yet another form of these instructions that may be present is a website address.

Thus, it will be appreciated that treatment with a CD47 blocking agent can cause an elevation within the cancer of the level at which at least one of the genes SPP1, CHIT1 and FCγR3A is expressed. In these patients, continued treatment with the CD47 blocking agent can be recommended. In other subjects, a different therapy should be adopted.

### Example 1: Gene Expression was Evaluated in a Clinical Trial Setting

Intratumoral injection of SIRPαFc (with IgG1 Fc) in percutaneously accessible tumors was performed in an investigational setting based on a modified 3+3 scheme with escalating doses sequentially through predefined levels of 1, 3, and 10 mg per injection. Injection frequency can be sequentially increased from single injections through 3 or 6 injections administered over 1 or 2 weeks. Dose expansion testing of the maximally assessed SIRPαFc dose and schedule proceeded with six 10 mg doses administered MWF over 2 weeks (induction therapy), in each of 6 cohorts.

Weekly continuation therapy beyond the initial 2 week induction therapy at investigator's discretion was incorporated into the study. Additional lesions can be injected beyond the 3 target lesions identified in induction therapy (rolling injections).

Composite Assessment of Index Lesion Severity (CAILS) scores for injected and non-injected lesions were assessed at the end of induction therapy and at later time points in some subjects. The CAILS score is a quantification of the severity of up to 5 index lesions: erythema, scaling, plaque elevation, and surface area. Severity is graded from 0 (none) to 8 (severe) for erythema and scaling; 0-3 for plaque elevation; and 0-9 for surface area (Olsen E A et al. 2011. JCO.). As a part of exploratory analyses, serial biopsies were collected to assess impact of SIRPαFc on the tumor microenvironment. Biopsies were collected per protocol prior to SIRPαFc treatment with a screening period of 14 days, at maximum induration, and end of induction therapy (7 days following the last injection). Adjacent, uninjected lesions were also biopsied at the same timepoints. For patients who went onto continuation therapy additional biopsies could be taken at the investigator's discretion.

### Example 2—Marker Gene Testing

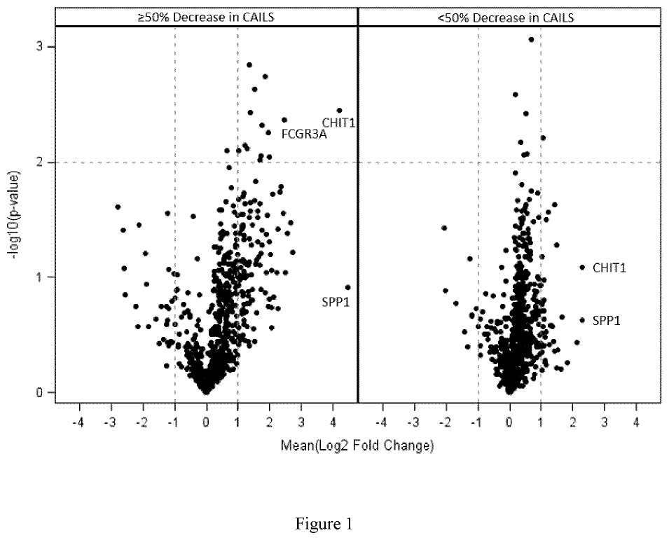

Total RNA from formalin fixed paraffin embedded (FFPE) biopsies was extracted. RNA quality and concentration were assessed. For each sample, mRNA transcript abundance was quantified using the NanoString nCounter Human PanCancer Immune Profiling Panel according to the manufacturer's protocol from 100 ng of total RNA. Normalization to housekeeping genes and subsequent analysis was performed using nSolver software (NanoString, Seattle). The log fold change is calculated for each patient pair from pre-treatment to maximum induration (MI) and is representative of a group of patients, the p-vale is the corresponding measurement of the significance within that group. Total counts were log 2 transformed. Fold-expression was determined as the ratio of matched on-treatment or end of treatment biopsies over pre-treatment. Group mean, standard deviation and p-values (for a null hypothesis that the ratio was 1) for each gene were calculated. Results were plotted as the log 10 p value by fold change. NanoString gene expression of biopsies at maximum induration (n=9) and end of treatment (n=12) in comparison to baseline indicate strong innate responses shortly after TTI-621 (SIRPαG1) exposure. Significant and strong upregulation of the genes CHIT1 FCγR2A (not shown) and FCγR3A was observed as well as strong upregulation of SPP1 (osteopontin) at maximum induration was observed. CHIT1 and SPP1 specifically upregulated to a greater extent in patients who had at least a 50% reduction in CAILS as compared to those who did not (FIG. 1). In the example below, dose was not taken into account.

### Example 3—In Vitro Evaluation of SPP1 Gene Expression

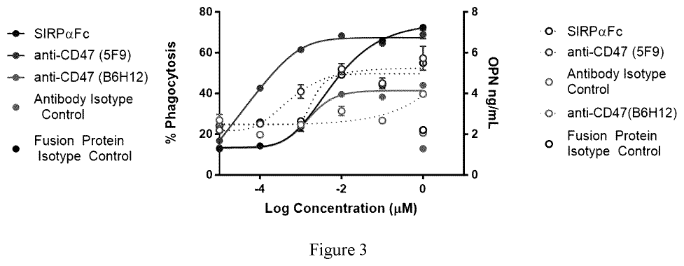

The protein product of SPP1 is osteopontin, a secreted cytokine. In vitro phagocytosis assays were set up to determine the effect of SIRPαFc on osteopontin production by macrophages. Phagocytosis assays were set up as described by Petrova et al. (2017). Briefly, healthy donor monocyte derived macrophages were primed with interferon-gamma prior to co-culture with a tumor cell line (Toledo) that had been exposed to various concentrations of SIRPαFc or isotype control. The log fold change is calculated for each patient pair from pre-treatment to maximum induration (MI) and is representative of a group of patients, the p-vale is the corresponding measurement of the significance within that group. After four hours of co-culture, the supernatant was collected for osteopontin evaluation by ELISA and phagocytosis was evaluated by flow cytometry. Osteopontin is increased following CD47 blockade with SIRPαFc, or anti-CD47 antibody clones 5F9 or B6H12 in a dose dependent manner (FIG. 3). These data suggest that production of osteopontin following SIRPαFc exposure in human tumors can be ascribed to, but not limited to, macrophages.

### Example 4—Evaluation of FcγR2a Gene Expression

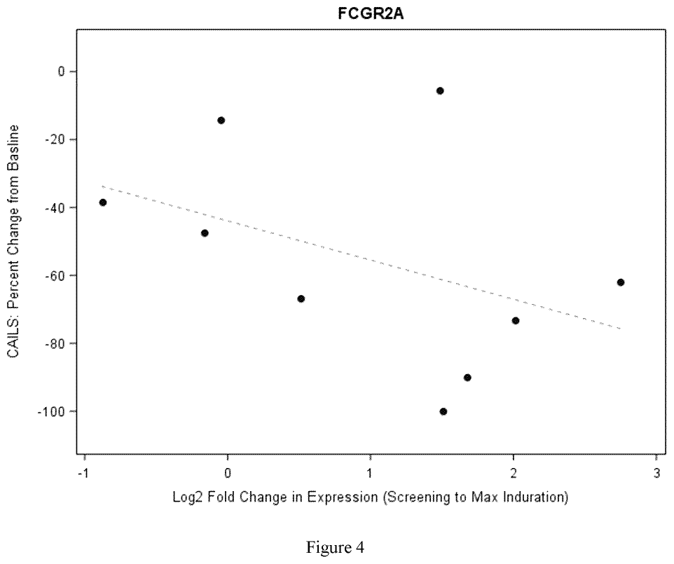

In vitro phagocytosis assays were set up to determine the effect of SIRPαFc on FcγR2a production by macrophages. Phagocytosis assays were set up as described by Petrova et al. (2017). Briefly, healthy donor monocyte derived macrophages were primed with interferon-gamma prior to co-culture with a tumor cell line (Toledo) that had been exposed to various concentrations of SIRPαFc or isotype control. After four hours of co-culture, the supernatant was collected for FcγR2a evaluation by ELISA and phagocytosis was evaluated by flow cytometry. As shown in FIG. 4, FcγR2a is increased following CD47 blockade with SIRPαFc, or anti-CD47 antibody clones 5F9 or B6H12 in a dose dependent manner. These data suggest that production of FcγR2a following SIRPαFc exposure in human tumors can be ascribed to, but not limited to, macrophages.

1. A method for treating a subject presenting with a CD47+ cancer, comprising administering a CD47 blocking agent to a subject determined to respond to the blocking agent with an elevated level of expression of a marker gene selected from SPP1, CHIT1, FCγR2A and FCγR3A.

2. (canceled)

3. A method of predicting responsiveness to therapy with a CD47 blocking agent of a cancer in a subject, the method comprising determining the expression level of one, two or all of the marker genes CHIT1, FCγR2A, FCγR3A and SPP1 in a sample of that cancer obtained from that subject relative to a control or normal level thereof, whereby elevated marker gene expression in a sample from a subject treated with the CD47 blocking agent predicts the cancer is responsive to therapy with that CD47 blocking agent.

4. The method according to claim 1, wherein the cancer is a blood cancer.

5. The method according to claim 1, wherein the cancer is a leukemia.

6. The method according to claim 1, wherein the cancer is a lymphoma.

7. The method according to claim 1, wherein the cancer is a myeloma.

8. The method according to claim 4, wherein the cancer is selected from the group consisting of Hodgkin's lymphoma, indolent and aggressive non-Hodgkin's lymphoma, Burkitt's lymphoma, follicular lymphoma, T cell lymphoma, mycosis fungoides, Sezary Syndrome, cutaneous T cell lymphoma (CTCL), promyelocytic leukemia, chronic and acute myeloid leukemia, acute and chronic lymphoid leukemia, multiple myeloma (MM), giant cell myeloma, heavy-chain myeloma, and light chain or Bence-Jones myeloma, diffuse large B-cell lymphoma (DLCBL), and T-cell acute lymphoblastic leukemia.

9. The method according to claim 1, wherein the CD47 blocking agent is SIRPαFc having an IgG1-based Fc region.

10. The method according to claim 1, wherein the CD47 blocking agent is SIRPαFc having an IgG4-based Fc region.

11. The method according to claim 1, wherein the marker comprises an elevated level of FCγR3A expression or of FCγR2A expression.

12. The method according to claim 1, wherein the marker comprises an elevated level of SPP1 expression.

13. The method according to claim 1, wherein the marker comprises an elevated level of CHIT1 expression.

14. The method according to claim 1, wherein the cancer is a solid tumour.

15. The method according to claim 1 wherein the cancer is an ovarian cancer.

16. The method according to claim 1 wherein the marker is a gene selected from CHIT1, FCγR2A, and FCγR3A, or an expression product thereof.

17. The method according to claim 16, wherein the marker is a gene selected from FCγR2A and FCγR3A, or an expression product thereof.

18. A kit useful to identify a cancer cell that will respond to treatment with a CD47 blocking agent, the kit comprising:
(a) means useful in determining the expression level of a marker gene selected from one or more of CHIT1, FCγR2A, FCγR3A, and SPP1; and
(b) instructions for the use thereof in determining that level, thereby to identify a candidate for therapy with that CD47 blocking agent.
